(19) United States
(12) Patent Application Publication
(10) Pub. No.: US 2018/0286511 A1
(43) Pub. Date: Oct. 4, 2018

(54) MEDICAL APPARATUS, INFORMATION PROCESSING APPARATUS, AND MEDICAL INFORMATION PROCESSING SYSTEM

(71) Applicant: Sony Olympus Medical Solutions Inc., Tokyo (JP)

(72) Inventors: Ryu SUKIGARA, Kanagawa (JP); Toshiya SUGAI, Tokyo (JP); Kiyoshi ICHIKAWA, Tokyo (JP); Hideki TASHIRO, Tokyo (JP)

(73) Assignee: Sony Olympus Medical Solutions Inc., Tokyo (JP)

(21) Appl. No.: 15/903,058

(22) Filed: Feb. 23, 2018

(30) Foreign Application Priority Data
Mar. 29, 2017 (JP) 2017-064194

(51) Int. Cl. G16H 40/40; G16H 20/10; G16H 40/60

(52) U.S. Cl. CPC G16H 40/40; G16H 10/60; G16H 40/60; G16H 20/10

(57) ABSTRACT

There is provided a medical apparatus including: a determination unit that determines a state that is a target of a notification; and a notification processing unit that causes, on a basis of a result of the determination, notifications of apparatus management information and medical apparatus notification information to be provided in a format in which an external apparatus is able to read the information, the apparatus management information being used for managing the medical apparatus, the medical apparatus notification information indicating content of a notification in the medical apparatus.

### CROSS REFERENCE TO RELATED APPLICATIONS

This application claims the benefit of Japanese Priority Patent Application JP 2017-064194 filed Mar. 29, 2017, the entire contents of which are incorporated herein by reference.

### BACKGROUND

The present disclosure relates to a medical apparatus, an information processing apparatus, a processing method, a program, and a medical information processing system.

Technologies related to systems for managing medical apparatuses have been developed. A technique described in JP2011-204205A is exemplified, for example, as a technique related to a system that manages medical equipment (corresponding to the medical apparatus; the same is true for the following description).

### SUMMARY

In a medical setting, various medical apparatuses are used. In addition, health care providers in medical settings are not necessarily familiar with the medical apparatuses. Therefore, a system capable of assisting a person, such as a health care provider, who uses a medical apparatus when malfunction of the medical apparatus occurs, for example, to improve convenience has been desired.

Here, the system in which the technique described in JP2011-204205A is used, for example, generates medical equipment information when a user, such as a health care provider, using the system operates an input terminal and manages the medical equipment by a management server processing the medical equipment information corresponding to medical equipment.

However, there is concern that the convenience of the user is damaged in a case in which the user, such as a health care provider, who uses the system has to operate the input terminal.

The present disclosure proposes a novel and improved medical apparatus, an information processing apparatus, a processing method, a program, and a medical information processing system capable of improving convenience of the person who uses the medical apparatus.

According to an embodiment, there is provided a medical apparatus including: a determination unit that determines a state that is a target of a notification; and a notification processing unit that causes, on a basis of a result of the determination, notifications of apparatus management information and medical apparatus notification information to be provided in a format in which an external apparatus is able to read the information, the apparatus management information being used for managing the medical apparatus, the medical apparatus notification information indicating content of a notification in the medical apparatus.

In addition, according to an embodiment of the present disclosure, there is provided an information processing apparatus including: an acquisition processing unit that performs processing of acquiring apparatus management information that is used for managing a medical apparatus and medical apparatus notification information that indicates content of a notification in the medical apparatus from the medical apparatus; and a transmission control unit that causes transmission information to be transmitted to a set predetermined transmission destination, the transmission information including the acquired apparatus management information and information based on the acquired medical apparatus notification information.

In addition, according to an embodiment of the present disclosure, there is provided a medical information processing system including: a medical apparatus; a first information processing apparatus; and a second information processing apparatus. The medical apparatus includes a first determination unit that determines a state that is a target of a notification, and a notification processing unit that causes, on a basis of a result of the determination, notifications of apparatus management information and medical apparatus notification information to be provided in a format in which an external apparatus is able to read the information, the apparatus management information being used for managing the medical apparatus, the medical apparatus notification information indicating content of a notification in the medical apparatus. The first information processing apparatus includes an acquisition processing unit that performs processing of acquiring the apparatus management information and the medical apparatus notification information from the medical apparatus, and a transmission control unit that causes transmission information to be transmitted to a set predetermined transmission destination, the transmission information including the acquired apparatus management information and information based on the acquired medical apparatus notification information. The second information processing apparatus includes a second determination unit that determines a type of the content of the notification in the medical apparatus on a basis of the transmission information in a case in which the transmission information is acquired, and a processing unit that performs processing corresponding to the determined type of the notification.

According to an embodiment of the present disclosure, it is possible to improve convenience of a person who uses the medical apparatus.

Note that the effects described above are not necessarily limitative. With or in the place of the above effects, there may be achieved any one of the effects described in this specification or other effects that may be grasped from this specification.

### BRIEF DESCRIPTION OF THE DRAWINGS

FIG. 1 is an explanatory diagram illustrating an example of a configuration of a medical information processing system according to an embodiment;

FIG. 2 is a block diagram illustrating an example of a configuration of a medical apparatus according to the embodiment;

FIG. 3 is a block diagram illustrating an example of a configuration of an information processing apparatus (first information processing apparatus) according to the embodiment;

FIG. 4 is an explanatory diagram illustrating an example of a hardware configuration of the information processing apparatus (first information processing apparatus) according to the embodiment;

FIG. 5 is an explanatory diagram for explaining an example of processing related to a processing method in the information processing apparatus (first information processing apparatus) according to the embodiment;

FIG. 6 is a block diagram illustrating an example of a configuration of an information processing apparatus (second information processing apparatus) according to the embodiment; and

FIG. 7 is an explanatory diagram illustrating an example of a hardware configuration of the information processing apparatus (second information processing apparatus) according to the embodiment.

### DETAILED DESCRIPTION OF THE EMBODIMENT(S)

Hereinafter, (a) preferred embodiment(s) of the present disclosure will be described in detail with reference to the appended drawings. Note that, in this specification and the appended drawings, structural elements that have substantially the same function and structure are denoted with the same reference numerals, and repeated explanation of these structural elements is omitted.

In addition, description will be given below in the following order.

1. Medical information processing system according to embodiment
2. Program according to embodiment

### Medical Information Processing System According to Embodiment

### [1] Configuration of Medical Information Processing System

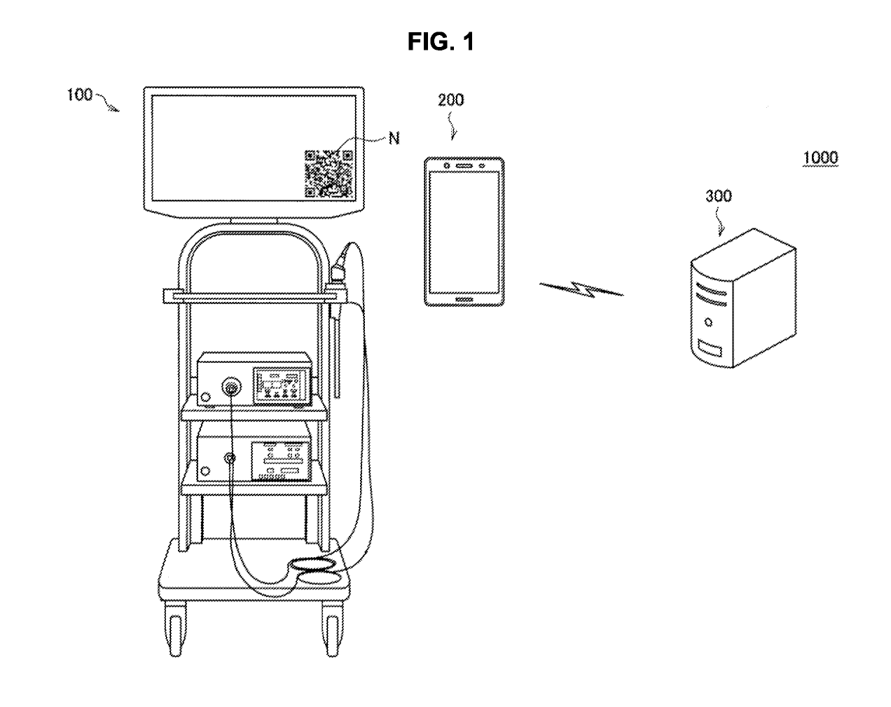

FIG. 1 is an explanatory diagram illustrating an example of a configuration of a medical information processing system 1000 according to an embodiment. The medical information processing system 1000 has, for example, a medical apparatus 100 (a medical apparatus or a medical apparatus system), an information processing apparatus 200 (first information processing apparatus), and an information processing apparatus 300 (second information processing apparatus).

FIG. 1 illustrates “an endoscope system that includes a processing device that is provided with a video scope with an imaging element at a tip end, a light source device, a processor and the like and a display device” as the medical apparatus 100. Note that the medical apparatus 100 is not limited to the endoscope system as illustrated in FIG. 1. For example, the medical apparatus 100 may be a medical observation apparatus such as a medical observation apparatus that is provided with an optical microscope or a medical observation apparatus that is provided with an imaging device that functions as an electronic imaging microscope. In addition, the medical apparatus 100 may be an arbitrary apparatus (or an arbitrary apparatus system that includes a plurality of apparatuses) that is used in the medical field and is capable of performing processing related to a processing method in the medical apparatus 100, which will be described later, such as “various medical image apparatuses (modality apparatuses) such as a biological information monitor or a navigation apparatus” or “various treatment apparatuses such as an electric scalpel, a bipolar, or an ultrasonic aspirator,” for example.

In addition, FIG. 1 illustrates a smartphone as the information processing apparatus 200. Note that the information processing apparatus 200 is not limited to a communication apparatus such as the smartphone illustrated in FIG. 1. For example, the information processing apparatus 200 may be an arbitrary apparatus capable of performing processing related to the processing method in the information processing apparatus 200, which will be described later, such as a computer including a personal computer (PC), or a tablet-type apparatus.

In addition, FIG. 1 illustrates a server as the information processing apparatus 300. Note that the information processing apparatus 300 is not limited to the server as illustrated in FIG. 1. For example, the information processing apparatus 300 may be an arbitrary apparatus capable of performing processing related to the processing method in the information processing apparatus 300, which will be described later, such as a computer including a PC or a tablet-type apparatus.

Note that the configuration of the medical information processing system according to the embodiment is not limited to the example illustrated in FIG. 1.

For example, the medical information processing system according to the embodiment may have a plurality of medical apparatuses 100. In a case in which the medical information processing system has a plurality of medical apparatuses 100, the processing related to the processing method in the medical apparatus 100, which will be described later, is individually performed in the plurality of medical apparatuses 100.

In addition, the medical information processing system according to the embodiment may have a plurality of information processing apparatuses 200. In a case in which the medical information processing system has a plurality of information processing apparatuses 200, the processing related to the processing method in the information processing apparatuses 200, which will be described later, is individually performed in the plurality of the information processing apparatuses 200.

In addition, the medical information processing system according to the embodiment may have a plurality of information processing apparatuses 300. In a case in which the medical information processing system has a plurality of information processing apparatuses 300, the processing related to the processing method in the information processing apparatuses 300, which will be described later, is individually performed in the plurality of the information processing apparatuses 300.

### [1-1] Medical Apparatus 100

### [1-1-1] Processing Related to Processing Method in Medical Apparatus 100

The medical apparatus 100 determines a state that is a target of a notification and provides a notification of information corresponding to a result of the determination of the state that is a target of a notification in a format in which an external apparatus such as the information processing apparatus 200 is able to read the information. Hereinafter, the information corresponding to the result of the determination of the state that is a target of a notification will be collectively referred to as “information corresponding to a result of the determination” in some cases. The notification in the format in which the external apparatus according to the embodiment is able to read the information is different from a notification by communication via a network. As the notification in the format in which the external apparatus according to the embodiment is able to read the information, for example, a notification that is made to be displayed on a display screen and a notification that is provided through one-way communication are exemplified as will be described later.

As a state that is a target of a notification to be determined by the medical apparatus 100, an error notification state in which a notification that an error has occurred is provided and a current state notification state in which a notification of a current state of the medical apparatus 100 is provided are exemplified, for example.

In addition, apparatus management information and medical apparatus notification information are exemplified, for example, as the information corresponding to the result of the determination according to the embodiment.

The apparatus management information according to the embodiment is data that is used for managing the medical apparatus 100. Identification information with which the medical apparatus 100 can be uniquely identified is exemplified, for example, as the apparatus management information. An apparatus ID of the medical apparatus 100 and a serial number of the medical apparatus 100 are exemplified, for example, as the identification information according to the embodiment.

Note that the apparatus management information according to the embodiment is not limited to the identification information described above. For example, the apparatus management information according to the embodiment may be arbitrary data (or a data group) with which the medical apparatus 100 can be uniquely specified and managed.

In addition, the medical apparatus notification information according to the embodiment is data that indicates content of the notification in the medical apparatus 100.

The content indicated by the medical apparatus notification information can change depending on the state determined as a target of a notification. In a case in which the state determined as a target of notification is the error notification state, the medical apparatus notification information includes one or two or more of an error number, a degree of urgency, an ID of a module or the like in which the error has occurred, and data that accompanies the error, for example. In addition, in a case in which the state determined as a target of a notification is the current state notification state, the medical apparatus notification information includes one or two or more of data that indicates a version of software or the like, data that indicates a cumulative operating time, data that indicates a parts use status (for example, data that indicates effective capacity of a battery, the number of times the battery is charged and discharged, an operating time of a lamp, and the like), and data that indicates an operation result of a self-diagnosis function, for example. Note that it is needless to say that the examples of the content indicated by the medical apparatus notification information are not limited to the examples described above.

Hereinafter, an example of a configuration of the medical apparatus 100 will be described, and an example of processing related to a processing method in the medical apparatus 100 will be described.

### [1-1-2] Configuration of Medical Apparatus 100

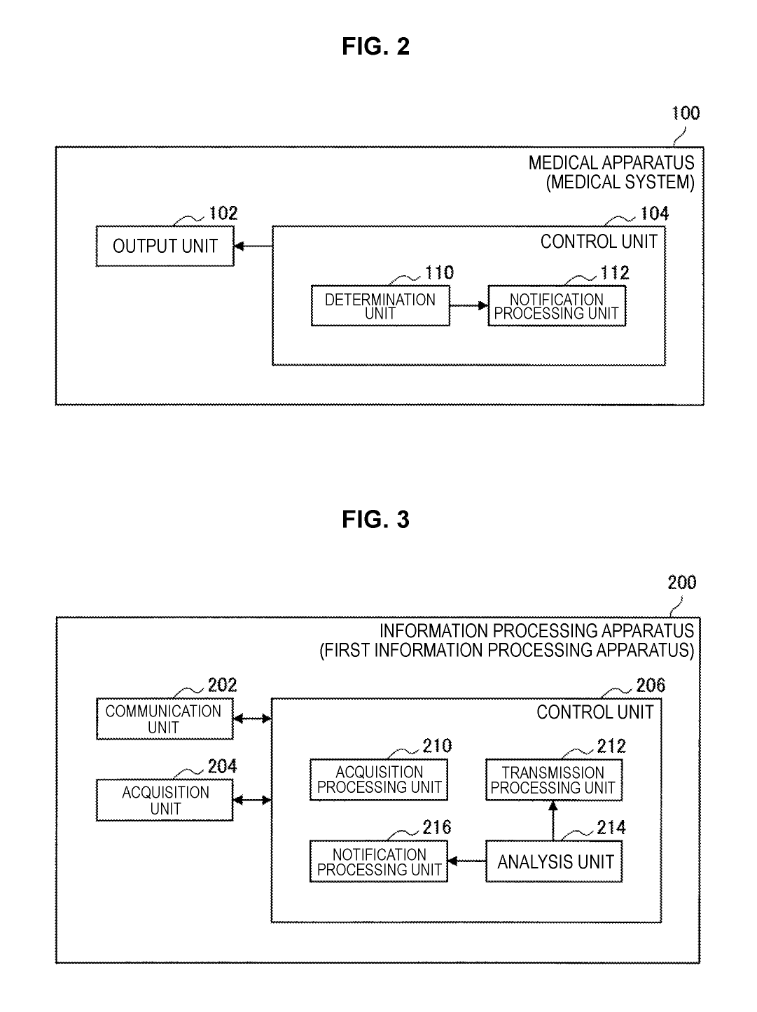

FIG. 2 is a block diagram illustrating an example of a configuration of the medical apparatus 100 according to the embodiment. The medical apparatus 100 includes an output unit 102 and a control unit 104, for example.

The medical apparatus 100 includes one or two or more processors (not illustrated) that include a computation circuit such as a micro processing unit (MPU) and the like, a read only memory (ROM; not illustrated), and a random access memory (RAM; not illustrated), for example. In addition, the medical apparatus 100 has a configuration for realizing the functions of the medical apparatus 100, for example. The medical apparatus 100 has a video scope, a light source device, a display device, and the like in a case in which the medical apparatus 100 is an endoscope system illustrated in FIG. 1, for example. For example, the medical apparatus 100 is driven by electric power that is supplied from an internal power source such as a battery provided in the medical apparatus 100, electric power that is supplied from an external power source connected thereto, or the like.

The processor (not illustrated) functions as the control unit 104 and plays a leading role in performing the processing related to the processing method in the medical apparatus 100, which will be described later. Note that the processing related to the processing method in the medical apparatus 100, which will be described later, may be performed by a processing circuit that is separate from the processor (not illustrated).

The ROM (not illustrated) stores various kinds of data such as programs that are used by the processor (not illustrated), data related to the processing related to the processing method in the medical apparatus 100, such as computation parameters, apparatus management information (which will be described later), and medical apparatus notification information (which will be described later), and the like. The RAM (not illustrated) temporarily stores the programs and the like that are executed by the processor (not illustrated).

The output unit 102 provides a notification of the information corresponding to the result of the determination to the outside. The notification from the output unit 102 is controlled by the control unit 104 (more specifically, the notification processing unit, which will be described later).

The output unit 102 provides the notification by causing the information corresponding to the result of the determination to be displayed on the display screen, for example.

In a case in which the notification of the information corresponding to the result of the determination is provided by causing the information to be displayed on the display screen, the output unit 102 includes a display device, for example. A liquid crystal display, an organic electro-luminescence (EL) display, a cathode ray tube (CRT) display, and the like are exemplified, for example, as the display device that is included in the output unit 102.

The output unit 102 provides the notification of the information corresponding to the result of the determination by causing the information to be displayed on the display screen by an arbitrary display method by which the notification can be recognized in the external apparatus, such as “causing a multidimensional code that indicates the information corresponding to the result of the determination, such as a two-dimensional code, to be displayed on the display screen of the display device as represented by the reference numeral N in FIG. 1” or “causing a character string that indicates content of the information corresponding to the result of the determination to be displayed on the display screen of the display device”, for example. In a case in which a character string that indicates content of the information corresponding to the result of the determination is displayed, the output unit 102 may cause a unique icon to be displayed along with the character string. It is possible to improve a rate of recognition of the notification in the external apparatus such as the information processing apparatus 200 by causing the unique icon to be displayed along with the character string.

Note that the display device is not limited to the example illustrated in FIG. 1 in the case in which the notification of the information corresponding to the result of the determination is provided by causing the information to be displayed on the display screen. The display device may be a display device capable of performing character display, such as one mounted on a calculator, in the case in which the notification of the information corresponding to the result of the determination is provided by causing the information to be displayed on the display screen, for example.

In addition, the output unit 102 may provide the notification of the information corresponding to the result of the determination by one-way communication, for example. The output unit 102 provides the notification of the information corresponding to the result of the determination by communication based on an arbitrary communication scheme by which the one-way communication can be performed, for example.

In the case in which the notification of the information corresponding to the result of the determination is provided by the one-way communication, the output unit 102 includes an output device capable of performing the one-way communication, for example. An output device that is compatible with an arbitrary communication scheme by which the one-way communication can be performed such as a communication device that is compatible with a predetermined communication standard such as RS232C, a communication device that is compatible with infrared communication, or a sound output device that outputs acoustic waves modulated by an arbitrary modulation scheme, such as a speaker, is exemplified, for example, as the output device that is included in the output unit 102. The acoustic waves output from the sound output device may be acoustic waves in an audible frequency band (10 to several tens of thousands of [Hz], for example) that can be heard by human ears, for example, or may be acoustic waves in a frequency band that inaudible by human ears, such as ultrasonic waves.

Here, it is possible to further improve security in the case in which the output unit 102 causes the notification of the information corresponding to the result of the determination to be provided by the one-way communication than in a case in which the output unit 102 causes the notification of the information corresponding to the result of the determination to be provided by two-way communication. Note that it is needless to say that the output unit 102 can provide the notification of the information corresponding to the result of the determination by the two-way communication.

The control unit 104 includes a processor (not illustrated) and the like, for example, and plays a role in controlling the entire medical apparatus 100. In addition, the control unit 104 includes a determination unit 110 (first determination unit) and a notification processing unit 112, for example, and plays a leading role in performing the processing related to the processing method in the medical apparatus 100.

The determination unit 110 determines a state that is a target of notification. As described above, the error notification state and the current notification state are exemplified, for example, as the state determined by the determination unit 110 as a target of a notification.

The determination unit 110 determines a case in which processing has not been performed normally when processing related to some functions is executed or a case in which an error has been discovered as a result of performing a self-diagnosis function, for example, as the error notification state. In addition, the determination unit 110 determines a case in which a predetermined operation such as an operation for maintenance has been detected as the current state notification state.

In a case of determining the state as a target of a notification, the determination unit 110 delivers the result of the determination to the notification processing unit 112.

The notification processing unit 112 provides notifications of the apparatus management information and the medical apparatus notification information in a format in which the external apparatus is able to read the information on the basis of the result of the determination delivered from the determination unit 110. The notification processing unit 112 causes the output unit 102 to output the apparatus management information and the medical apparatus notification information corresponding to the result of the determination delivered from the determination unit 110, for example.

In addition, the notification processing unit 112 may encrypt one of or both the apparatus management information and the medical apparatus notification information and then causes notifications thereof to be provided. The notification processing unit 112 performs the encryption with an encryption key that can be decoded by one of or both the information processing apparatus 200 and the information processing apparatus 300, for example, (a public key that is shared by one of or both the information processing apparatus 200 and the information processing apparatus 300, a public key of the information processing apparatus 200, or a public key of the information processing apparatus 300, for example).

In a case in which the output unit 102 is made to provide the notification of the information corresponding to the result of the determination by causing the information to be displayed on the display screen, for example, the notification processing unit 112 generates a two-dimensional code (an example of the multidimensional code; the same is true to the following description) that indicates the information corresponding to the result of the determination and causes the generated two-dimensional code to be displayed on the display device that is included in the output unit 102. The notification processing unit 112 causes the generated two-dimensional code to be displayed on the display device by delivering a control signal for controlling display and a signal that indicates the generated two-dimensional code, for example, to the display device.

Note that the notification processing unit 112 can also cause the character string or the like that indicates the information corresponding to the result of the determination to be displayed on the display device included in the output unit 102 as described above.

In addition, in the case in which the output unit 102 is made to provide the notification of the information corresponding to the result of the determination by the one-way communication, for example, the notification processing unit 112 causes the information corresponding to the result of the determination to be transmitted by the one-way communication that is a communication scheme with which the communication device is compatible by delivering the control signal for controlling communication and the information corresponding to the result of the determination to a communication device (an example of the output device) that is included in the output unit 102, for example. In addition, in a case in which the notification of the information corresponding to the result of the determination is made to be provided by the one-way communication, the notification processing unit 112 may cause a sound output device (an example of the output device) that is included in the output unit 102 to output acoustic waves obtained by modulating the information corresponding to the result of the determination by delivering a signal obtained by modulating the information corresponding to the result of the determination in an arbitrary modulation scheme, for example, to the sound output device.

Note that the notification processing unit 112 can also cause the notification of the information corresponding to the result of the determination to be provided by the two-way communication as described above.

The medical apparatus 100 determines the state as a target of a notification with the configuration illustrated in FIG. 2, for example, and provides a notification of the information corresponding to the result of the determination of the state as a target of a notification.

Note that the configuration of the medical apparatus 100 is not limited to the example illustrated in FIG. 2.

For example, the medical apparatus 100 can include one of or both the determination unit 110 and the notification processing unit 112 illustrated in FIG. 2 separately from the control unit 104 (that is realized by a different processing circuit, for example).

In addition, the medical apparatus 100 may not include the output unit 102 in a case in which the notification of the information corresponding to the result of the determination is provided via an external output device that has functions and a configuration that are similar to those of the output unit 102, for example.

### [1-2] Information Processing Apparatus 200

### [1-2-1] Processing Related to Processing Method in Information Processing Apparatus 200

The information processing apparatus 200 acquires the information corresponding to the result of the determination, a notification of which has been provided from the medical apparatus 100, for example, and transmits transmission information based on the acquired information corresponding to the result of the determination to a set predetermined transmission destination.

In a case in which the notification of the information corresponding to the result of the determination is provided by causing the information to be displayed on the display screen, the information processing apparatus 200 acquires the information corresponding to the result of the determination by capturing an image by an imaging device.

In addition, in a case in which the notification of the information corresponding to the result of the determination is provided by the one-way communication (or the two-way communication), the information processing apparatus 200 acquires the information corresponding to the result of the determination by receiving the information corresponding to the result of the determination by a device that is compatible with the communication. A communication device, a microphone, or the like is exemplified, for example, as the device that is compatible with the communication.

For example, the information corresponding to the result of the determination that is acquired as described above is recorded in a recording medium such as a storage unit (which will be described later) that is provided in the information processing apparatus 200, for example.

The transmission information according to the embodiment includes the apparatus management information acquired from the medical apparatus 100 and information based on the medical apparatus notification information acquired from the medical apparatus 100, for example.

The medical apparatus notification information acquired from the medical apparatus 100 is exemplified, for example, as the information based on the medical apparatus notification information according to the embodiment.

In addition, the information based on the medical apparatus notification information according to the embodiment may be information that indicates a result of analyzing the medical apparatus notification information acquired from the medical apparatus 100. Data that indicates a primary strategy for handling an error (for example, an ID or the like that is associated with the primary handling strategy) is exemplified, for example, as the information that indicates a result of analyzing the medical apparatus notification information. The information processing apparatus 200 analyzes the medical apparatus notification information acquired by an analysis unit, which will be described later, for example. Note that the analysis of the medical apparatus notification information may be performed by an external apparatus of the information processing apparatus 200.

In a case in which the information corresponding to the result of the determination that is obtained from the medical apparatus 100 is encrypted (in a case in which one of or both the apparatus management information and the medical apparatus notification information are encrypted), the information processing apparatus 200 may decode the encrypted information corresponding to the result of the determination or may not decode the encrypted information corresponding to the result of the determination. The transmission information includes the decoded information corresponding to the determination result or information based on the decoded information corresponding to the result of the determination in a case in which the information processing apparatus 200 decodes the encrypted information corresponding to the result of the determination. In addition, the transmission information includes the encrypted information corresponding to the result of the determination in a case in which the information processing apparatus 200 does not decode the encrypted information corresponding to the result of the determination.

Note that the information included in the transmission information is not limited to the examples described above. For example, the information processing apparatus 200 can also transmit transmission information that further includes additional information that is added by the information processing apparatus 200.

One or two or more of data that is set in advance for the information processing apparatus 200, such as identification information that indicates the information processing apparatus 200, position information that indicates the position of the information processing apparatus 200, and information that is set by an operation of a user of the information processing apparatus 200 are exemplified, for example, as the additional information according to the embodiment. One or two or more of data that indicates an owner who owns the medical apparatus 100 (an ID or the like that indicates the owner, for example), data that indicates an installation location of the medical apparatus 100 (an ID or the like that indicates facilities where the apparatus is installed, for example), data that indicates a comment of a user or the like of the medical apparatus 100, data that indicates a sales agent that has sold the medical apparatus 100 (an ID or the like that indicates the sales agent, for example) are exemplified, for example, as the information set by the operation of the user of the information processing apparatus 200. Note that it is needless to say that the examples of the additional information according to the embodiment are not limited to the examples described above.

The information processing apparatus 200 transmits the transmission information as described above, for example, to the set predetermined transmission destination.

An apparatus such as the information processing apparatus 300, a mail address, a uniform resource locator (URL), and the like are exemplified, for example, as the set predetermined transmission destination. The information processing apparatus 300 specifies the transmission destination with reference to information that indicates the transmission destination of the transmission information (for example, data or the like that indicates an Internet protocol (IP) address of the information processing apparatus 300), for example. The information that indicates the transmission destination is stored in a recording medium such as a storage unit (which will be described later), for example.

In a case in which a plurality of predetermined transmission destinations are present, such as a case in which a plurality of information processing apparatuses 300 are present, the information processing apparatuses 200 transmit the transmission information to the transmission destinations corresponding to the medical apparatus 100 that has acquired the information corresponding to the result of the determination. In the case in which the plurality of predetermined transmission destinations are present, the information that indicates the transmission destinations is associated with the identification information of the medical apparatus 100 and is stored in the recording medium, for example.

Note that the processing related to the processing method in the information processing apparatus 200 is not limited to the examples described above.

As described above, the information processing apparatus 200 may perform the processing of analyzing the medical apparatus notification information acquired from the medical apparatus 100.

In one example of the analysis processing, the information processing apparatus 200 specifies a primary strategy for handling an error from a combination of an apparatus ID, a serial number, an error number, a degree of urgency, an ID of a module or the like in which the error has occurred, data that accompanies the error, and the like, for example, and generates data that indicates the specified primary handling strategy. The information processing apparatus 200 specifies the primary strategy for handling the error with reference to the information that indicates the primary strategy for handling the error, which has been stored in a storage unit (which will be described later), or information that indicates the primary strategy for handling the error that is stored in an external apparatus, such as a server, for example. Note that the analysis processing is not limited to the examples as described above and that the information processing apparatus 200 may analyze the medical apparatus notification information by an arbitrary algorithm.

In addition, the information processing apparatus 200 may further provide a notification of information based on a result of the analysis in the case in which the analysis processing is performed. The information processing apparatus 200 provides the notification of the information based on the result of the analysis as a visual notification by causing a character string or an image that indicates the result of the analysis (an example of the information based on the result of the analysis) to be displayed on the display screen, an audible notification by causing sound that indicates the result of the analysis (another example of the information based on the result of the analysis) to be output from a sound output device such as a speaker, a combination thereof, or the like, for example. A person who receives the notification from the information processing apparatus 200 by the notification of the information based on the analysis result being provided, for example, can immediately perform the primary strategy for handling the error that has occurred in the medical apparatus 100.

In addition, the information processing apparatus 200 may provide a notification of response information in a case in which the response information that indicates a response to the transmission information is received from the transmission destination of the transmission information. The information processing apparatus 200 provides the notification of the response information by a visual notification by causing a character string or an image that indicates content of the response (an example of the response information) to be displayed on the display screen, an audible notification by causing sound that indicates content of the response (another example of the response information) to be output from a sound output device such as a speaker, a combination thereof, or the like, for example.

Hereinafter, an example of a configuration of the information processing apparatus 200 will be described, and an example of the processing related to the processing method in the information processing apparatus 200 will be described.

### [1-2-2] Configuration of Information Processing Apparatus 200

FIG. 3 is a block diagram illustrating an example of a configuration of the information processing apparatus 200 (first information processing apparatus) according to the embodiment. The information processing apparatus 200 includes a communication unit 202, an acquisition unit 204, and a control unit 206, for example.

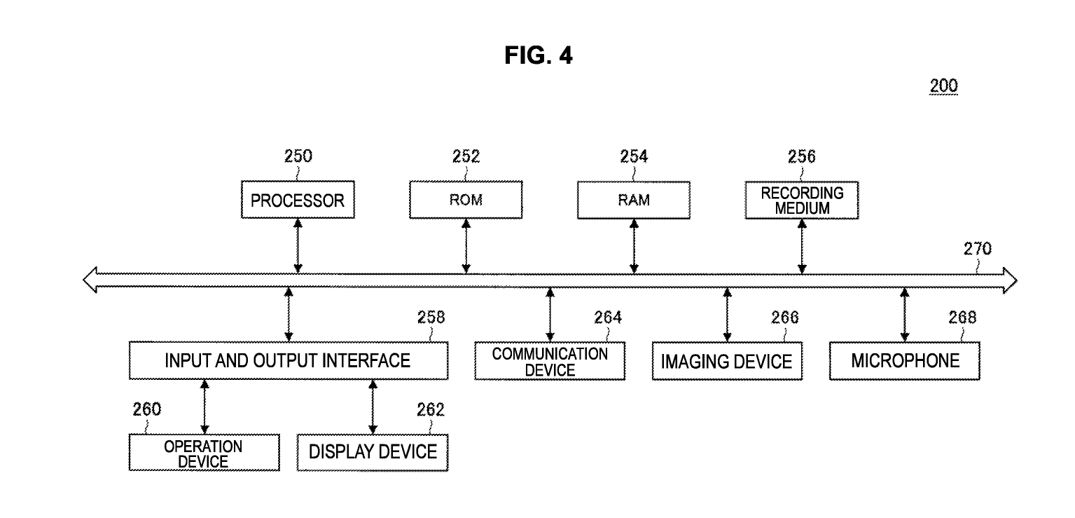

FIG. 4 is an explanatory diagram illustrating an example of a hardware configuration of the information processing apparatus 200 (first information processing apparatus) according to the embodiment. The information processing apparatus 200 includes a processor 250, a ROM 252, a RAM 254, a recording medium 256, an input and output interface 258, an operation device 260, a display device 262, a communication device 264, an imaging device 266, and a microphone 268, for example. In addition, the information processing apparatus 200 connects the respective components by a bus 270 that is a transmission path of data, for example. In addition, the information processing apparatus 200 is driven by electric power supplied from an internal power source such as a battery that is provided in the information processing apparatus 200, electric power supplied from an external power source connected thereto, or the like, for example.

The processor 250 includes, for example, one or two or more processors, including computation circuits such as an MPU, various processing circuits, and the like and functions as the control unit 206 that controls the overall information processing apparatus 200. In addition, the processor 250 plays roles of an acquisition processing unit 210, a transmission processing unit 212, an analysis unit 214, and a notification processing unit 216, which will be described later, for example, in the information processing apparatus 200. Note that one or two or more of the acquisition processing unit 210, the transmission processing unit 212, the analysis unit 214, and the notification processing unit 216 may be dedicated (or general-purpose) circuits (for example, processors that are separate from the processor 250).

The ROM 252 stores control data such as programs and computation parameters that are used by the processor 250. The RAM 254 temporarily stores the programs and the like that are executed by the processor 250, for example.

The recording medium 256 functions as a storage unit (not illustrated) and stores various kinds of data such as data related to the processing related to the processing method in the information processing apparatus 200, such as information that indicates the transmission destination, and a variety of kinds of applications, for example. Here, a magnetic recording medium such as a hard disk and a non-volatile memory such as a flash memory are exemplified, for example, as the recording medium 256. In addition, the recording medium 256 may be able to be attached to and detached from the information processing apparatus 200.

The input and output interface 258 connects the operation device 260 and the display device 262, for example. The operation device 260 functions as an operation unit (not illustrated) while the display device 262 functions as a display unit (not illustrated). Here, a universal serial bus (USB) terminal, a digital visual interface (DVI) terminal, a high-definition multimedia interface (HDMI) (registered trademark) terminal, various processing circuits, and the like are exemplified, for example, as the input and output interface 258.

In addition, the operation device 260 is provided on the information processing apparatus 200, for example, and is connected to the input and output interface 258 inside the information processing apparatus 200. A button, a direction key, a rotation-type selector such as a jog dial, a combination thereof, and the like are exemplified, for example, as the operation device 260.

In addition, the display device 262 is provided on the information processing apparatus 200, for example, and is connected to the input and output interface 258 inside the information processing apparatus 200. A liquid crystal display, an organic EL display, and the like are exemplified, for example, as the display device 262.

Note that it is needless to say that the input and output interface 258 can connect to an external device such as an operation device outside the information processing apparatus 200 (for example, a keyboard, a mouse, or the like), an external display device, or the like. In addition, the display device 262 may be a device that allows for display and user's operation, such as a touch panel, for example.

The communication device 264 is a communication mechanism provided in the information processing apparatus 200. The communication device 264 functions as the communication unit 202 for performing wireless or wired communication with an external apparatus such as the information processing apparatus 300, for example, via a network (or directly), for example. In addition, the communication device 264 may function as the acquisition unit 204 for acquiring the information corresponding to the result of the determination, the notification of which is provided from the medical apparatus 100, for example.

Here, a communication antenna and a radio frequency (RF) circuit (wireless communication), an IEEE 802.15.1 port and a transmission and reception circuit (wireless communication), an IEEE 802.11 port and a transmission and reception circuit (wireless communication), a local area network (LAN) terminal and a transmission and reception circuit (wired communication), and the like are exemplified, for example, as the communication device 264. In addition, the communication device 264 may have an arbitrary configuration that is compatible with the network. In addition, a wired network such as a LAN or a wide area network (WAN), a wireless network such as a wireless local area network (WLAN), the Internet using a communication protocol such as a transmission control protocol/Internet protocol (TCP/IP), and the like are exemplified, for example, as the network according to the embodiment.

The imaging device 266 generates a captured image (a moving image or a stationary image) by image capturing. The imaging device 266 functions as the acquisition unit 204 that acquires the information corresponding to the result of the determination in a case in which the notification of the information corresponding to the result of the determination is provided by causing the information to be displayed on the display screen, for example.

The imaging device 266 includes a lens/imaging element and a signal processing circuit, for example. The lens/imaging element includes a lens of an optical system and an image sensor using a plurality of imaging elements such as complementary metal oxide semiconductors (CMOSs), for example. The signal processing circuit includes an automatic gain control (AGC) circuit and an analog-to-digital converter (ADC), for example, and coverts an analog signal generated by the imaging elements to a digital signal (image data). In addition, the signal processing circuit performs various kinds of processing related to a RAW development, for example. Further, the signal processing circuit may perform various kinds of signal processing such as while balance correction processing, color tone correction processing, gamma correction processing, YCbCr conversion processing, and edge emphasis processing, for example.

The microphone 268 (sound collecting device) generates sound data from collected sound. The microphone 268 functions as the acquisition unit 204 that acquires the information corresponding to the result of the determination in a case in which the notification of the information corresponding to the result of the determination is provided by acoustic waves modulated by an arbitrary modulation scheme (an example of a case in which the notification of the information corresponding to the result of the determination is provided by the one-way communication), for example. The microphone 268 may have directionality or may not have directionality.

The information processing apparatus 200 has the hardware configuration illustrated in FIG. 4, for example. Note that the hardware configuration of the information processing apparatus 200 according to the embodiment is not limited to the configuration illustrated in FIG. 4.

For example, the information processing apparatus 200 may not include the communication device 264 in a case of performing communication with an external apparatus or the like via an external communication device connected thereto. In addition, the communication device 264 may have a configuration capable of performing communication with one or two or more external apparatuses by a plurality of communication schemes.

In addition, the information processing apparatus 200 may not include the imaging device 266 in a case in which the imaging function is not provided or in a case in which the imaging function is provided by an external imaging device connected thereto.

In addition, the information processing apparatus 200 may not include the microphone 268 in a case in which a sound collecting function is not provided or in a case in which the sound collecting function is provided by an external microphone connected thereto.

In addition, the information processing apparatus 200 can employ a configuration in which a part or an entirety of the recording medium 256, the operation device 260, and the display device 262 are not provided, for example.

In addition, the information processing apparatus 200 may include a position sensor capable of detecting a position by an arbitrary method, such as a global navigation satellite system (GNSS) device, for example.

In addition, the information processing apparatus 200 may include a sound output device such as a speaker, for example.

In addition, the information processing apparatus 200 can employ a configuration in accordance with an application example of the information processing apparatus 200, for example.

In addition, a part or an entirety of the configuration (or a configuration according to a modification example) illustrated in FIG. 4, for example, may be realized by one or two or more integrated circuits (ICs).

Referring again to FIG. 3, an example of a configuration of the information processing apparatus 200 will be described. The communication unit 202 is a communication mechanism provided in the information processing apparatus 200 and performs wireless or wired communication with an external apparatus such as the information processing apparatus 300, for example, via a network (or directly). In addition, communication by the communication unit 202 is controlled by the control unit 206, for example.

Here, although a communication antenna and an RF circuit, a LAN terminal and a transmission and reception circuit, and the like are exemplified, for example, as the communication unit 202, the configuration of the communication unit 202 is not limited to that described above. For example, the communication 202 can employ a configuration that compatible with an arbitrary standard capable of performing communication with the USB terminal, the transmission and reception circuit, and the like and an arbitrary configuration capable of performing communication with an external apparatus via a network. In addition, the communication unit 202 may have a configuration capable of performing communication with one or two or more external apparatuses and the like by a plurality of communication schemes.

In addition, the communication unit 202 can function as the acquisition unit 204 for acquiring the information corresponding to the result of the determination, the notification of which is provided from the medical apparatus 100.

The acquisition unit 204 acquires the information corresponding to the result of the determination, the notification of which is provided from the medical apparatus 100. In addition, operations of the acquisition unit 204 are controlled by the control unit 206, for example.

The imaging device 266 and the microphone 268 are exemplified, for example, as the acquisition unit 204. In addition, the acquisition unit 204 can function as the communication unit 202.

The control unit 206 includes the processor 250, for example, and plays a role in controlling the entire information processing apparatus 200. In addition, the control unit 206 includes the acquisition processing unit 210, the transmission processing unit 212, the analysis unit 214, and the notification processing unit 216, for example, and plays a leading role in performing the processing related to the processing method in the information processing apparatus 200.

The acquisition processing unit 210 performs the processing of acquiring the information corresponding to the result of the determination from the medical apparatus 100.

In a case in which the notification of the information corresponding to the result of the determination is provided by causing the information to be displayed on the display screen, for example, the acquisition processing unit 210 controls image capturing by the imaging device 266 that is included in the acquisition unit 204 and causes the imaging device 266 to capture an image. For example, a captured image that includes a two-dimensional code indicating the information corresponding to the result of the determination is obtained by the display screen of the medical apparatus 100 illustrated in FIG. 1 being imaged, and as a result, the information corresponding to the result of the determination is acquired by the information processing apparatus 200 from the medical apparatus 100.

In a case in which the notification of the information corresponding to the result of the determination is provided by acoustic waves (an example of a case in which the notification of the information corresponding to the result of the determination is provided by one-way communication), for example, the acquisition processing unit 210 controls the microphone 268 that is included in the acquisition unit 204 and causes the microphone 268 to start sound collection. By the acoustic waves output from the medical apparatus 100 being included in the sound collected by the microphone 268, the information corresponding to the result of the determination is acquired by the information processing apparatus 200 from the medical apparatus 100.

In addition, in a case in which the notification of the information corresponding to the result of the determination is provided by one-way communication based on a communication scheme other than the acoustic waves, for example, the acquisition processing unit 210 controls the communication device of the communication unit 202 that functions as the acquisition unit 204 and causes the communication device to receive the information corresponding to the result of the determination. By the communication device receiving the information corresponding to the result of the determination, the information corresponding to the result of the determination is acquired by the information processing apparatus 200 from the medical apparatus 100.

The transmission processing unit 212 causes the transmission information to be transmitted to the set predetermined transmission destination.

The transmission processing unit 212 generates the transmission information on the basis of the information corresponding to the result of the determination that has been received by the communication unit 202, for example, and causes the communication unit 202 to transmit the generated transmission information to the predetermined transmission destination. As described above, the transmission information that the transmission processing unit 212 is made to transmit can include one of or both the information that indicates the result of analysis of the acquired medical apparatus notification information and the additional information. The information that indicates the result of the analysis can be acquired from the acquisition unit 214, for example.

Note that the transmission processing unit 212 can transmit the information corresponding to the determination result itself, which has been received by the communication unit 202, as the transmission information.

The analysis unit 214 performs the analysis processing as described above and analyzes the acquired medical apparatus notification information. The result of the analysis by the analysis unit 214 is delivered to the transmission processing unit 212 and the notification processing unit 216, for example.

The notification processing unit 216 causes one of or both the notifications of the information based on the result of the analysis by the analysis unit 214 and the response information received by the communication unit 202 to be respectively provided. As described above, the notification processing unit 216 causes one of or both the notifications of the information based on the result of the analysis and the response information to be provided by a visual notification by controlling the display device 262, an audible notification by controlling a sound output device (not illustrated), a combination thereof, or the like, for example.

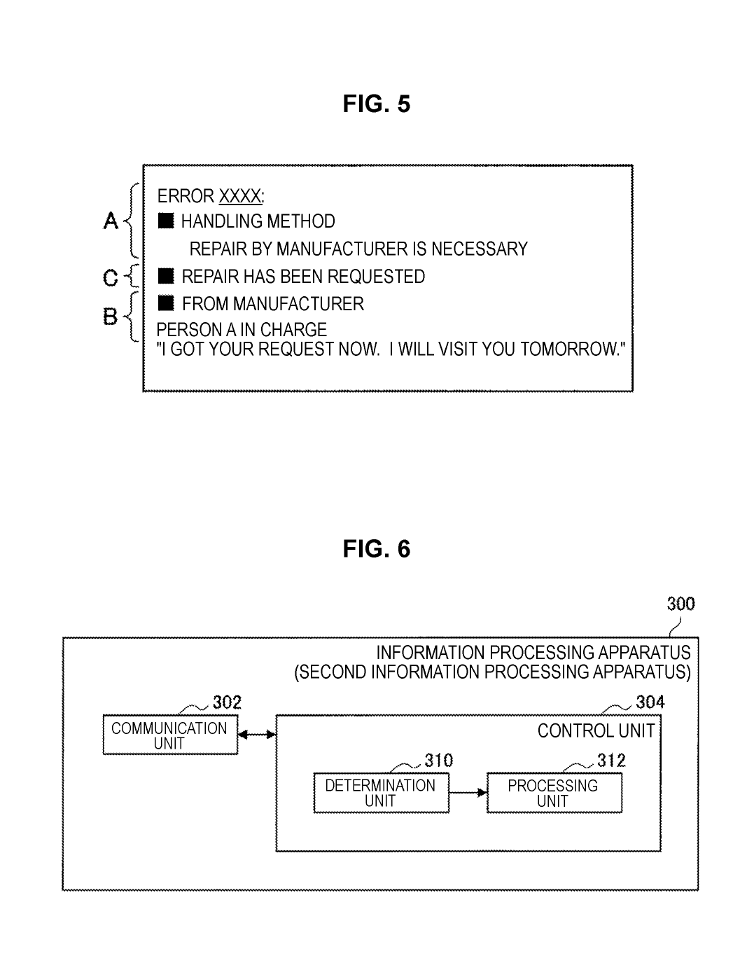

FIG. 5 is an explanatory diagram for explaining an example of the processing related to the processing method in the information processing apparatus 200 (first information processing apparatus) according to the embodiment. FIG. 5 illustrates an example of display on the display screen in a case in which the notification processing unit 216 causes both the information based on the result of the analysis and the response information to be displayed on the display screen of the display device 262.

A illustrated in FIG. 5 illustrates an example of content indicated by the information based on the result of the analysis, and B illustrated in FIG. 5 illustrates an example of content indicated by the response information. In addition, the notification processing unit 216 may further provide a notification of the content of the measure that the information processing apparatus 200 has employed as illustrated by C in FIG. 5.

Note that it is needless to say that the example of the notification provided by the notification processing unit 216 is not limited to the example illustrated in FIG. 5.

The information processing apparatus 200 acquires the information corresponding to the result of the determination, the notification of which has been provided from the medical apparatus 100, with the configuration illustrated in FIG. 3, for example, and transmits the transmission information based on the acquired information corresponding to the result of the determination to the set predetermined transmission destination. In addition, the information processing apparatus 200 performs other kinds of processing related to the processing method in the information processing apparatus 200, such as analysis processing, with the configuration illustrated in FIG. 3, for example.

Note that the configuration of the information processing apparatus 200 is not limited to the example illustrated in FIG. 3.

For example, the information processing apparatus 200 can include one or two or more of the acquisition processing unit 210, the transmission processing unit 212, the analysis unit 214, and the notification processing unit 216 illustrated in FIG. 3 separately from the control unit 206 (realized by different processing circuits, for example).

In addition, the information processing apparatus 200 may not include the communication unit 202 in a case in which the information processing apparatus 200 performs communication with an external apparatus via an external communication device that has functions and a configuration similar to those of the communication unit 202, for example.

In addition, the information processing apparatus 200 may not include the acquisition unit 204 in a case in which the information corresponding to the result of the determination is acquired from the medical apparatus 100 via an external device that has functions and a configuration similar to those of the acquisition unit 204 or a case in which the communication unit 202 functions as the acquisition unit 204, for example.

### [1-3] Information Processing Apparatus 300

### [1-3-1] Processing Related to Processing Method in Information Processing Apparatus 300

The information processing apparatus 300 determines a type of content of the notification in the medical apparatus 100 on the basis of the transmission information and performs processing corresponding to the determined type of the notification in the case in which the transmission information has been acquired, for example.

The information processing apparatus 300 acquires the transmission information by communication via the network (or directly) with the information processing apparatus 200, for example. In addition, the information processing apparatus 300 can also acquire the transmission information by an arbitrary method by which the transmission information can be obtained, such as acquisition of the transmission information attached to a mail from a mail server (not illustrated), for example.

In a case in which the acquired transmission information includes the information corresponding to the result of the determination that has been acquired from the medical apparatus 100 and the information corresponding to the result of the determination has been encrypted (a case in which one of or both the apparatus management information and the medical apparatus notification information have been encrypted), the information processing apparatus 300 decodes the encrypted information corresponding to the result of the determination and performs processing thereon.

The information processing apparatus 300 specifies the medical apparatus 100 that has provided the notification, on the basis of the apparatus management information included in the transmission information, for example. The information processing apparatus 300 specifies the medical apparatus 100 with an apparatus ID and a serial number that are indicated by the apparatus management information, for example.

Note that the specification of the medical apparatus 100 that has provided the notification can include specification of an installation location of the medical apparatus 100 in addition to the specification of the medical apparatus 100. The information processing apparatus 300 specifies the installation location of the medical apparatus 100 with reference to “a database (or a table) in which the apparatus ID and the serial number of the medical apparatus 100 are associated with data that indicates the installation location”, for example. In addition, the information processing apparatus 300 may specify the installation location of the medical apparatus 100 by position information of the information processing apparatus 200 that is indicated by the additional information included in the transmission information and information set by a user's operation performed on the information processing apparatus 200 that is indicated by the additional information, for example.

In addition, the information processing apparatus 300 determines a type of content of the notification in the specified medical apparatus 100 by information based on the medical apparatus notification information that is included in the transmission information, for example. An error occurrence notification in which a notification that an error has occurred in the medical apparatus 100 is provided (that the medical apparatus 100 is in an error notification state) and a current state report notification in which a notification of the current state of the medical apparatus 100 is provided (that the medical apparatus 100 is in a current state notification state) are exemplified as the type of the content of the notification that is determined by the information processing apparatus 300, for example.

In a case in which the type of the content of the notification is determined to be an error occurrence notification, for example, the information processing apparatus 300 performs processing of providing a notification that an error has occurred in the medical apparatus 100 to a contact destination corresponding to the specified medical apparatus 100. The information processing apparatus 300 provides notification of the medical apparatus 100 in which the error has occurred and the content of the error that has occurred, for example. Note that the content as a target of the notification provided by the information processing apparatus 30 is not limited to the examples described above. For example, the information processing apparatus 300 may further provide a notification of a strategy for handling an error or the like.

The information processing apparatus 300 provides the notification to the contact destination by an arbitrary method by which the notification to the contact destination corresponding to the specified medical apparatus 100 can be provided, such as a notification by characters or an image using an electronic mail or a sound notification using a telephone, for example.

A sales agent of the medical apparatus 100, a manufacturer of the medical apparatus 100, and the like are exemplified, for example, as the contact destination corresponding to the medical apparatus 100.

The information processing apparatus 300 specifies information that indicates the contact destination corresponding to the medical apparatus 100 with reference to “a database (or a table) in which the apparatus ID and the serial number of the medical apparatus 100 and the information that indicates the contact destination are associated”, for example. Data that indicates a mail address of the sales agent or the like and data that indicates a phone number of the sales agent or the like are exemplified, for example, as the information that indicates the contact destination. The information that indicates the contact destination is stored in a recording medium such as a storage unit (which will be described later) provided in the information processing apparatus 300, for example.

Then, the information processing apparatus 300 provides the notification to the contact destination corresponding to the specified medical apparatus 100 on the basis of the information that indicates the specified contact destination. In a case in which the notification has been provided to the contact destination, the information processing apparatus 300 may transmit response information that indicates content corresponding to the provided notification to the information processing apparatus 200 that has transmitted the transmission information.

In addition, the information processing apparatus 300 can also perform “processing of providing a notification of a part or an entirety of the content of the notification indicated by the transmission information, the determined type of the content of the notification, the content of the notification provided to the contact destination, and the like to a supervisor or an operator of the information processing apparatus 300”, for example. The information processing apparatus 300 provides the notification to the supervisor or the operator of the information processing apparatus 300 by an arbitrary method the notification can be provided to the supervisor or the operator of the information processing apparatus 300, such as display on the display screen, sound output from a sound output device such as a speaker, or a combination thereof, for example. In a case in which the notification to the supervisor or the operator of the information processing apparatus 300 has been provided, the information processing apparatus 300 may transmit response information that indicates the content corresponding to the provided notification to the information processing apparatus 200 that has transmitted the transmission information. The response information may include content that is input by the supervisor or the operator by using an operation device or the like.

In a case in which the information processing apparatus 300 performs the processing of providing the notification to the supervisor or the operator of the information processing apparatus 300 described above, the notification to the contact destination is manually provided by the supervisor, the operator, or the like of the information processing apparatus 300, for example.

In addition, in a case in which the type of the content of the notification is determined to be the current state report notification, for example, the information processing apparatus 300 performs processing of updating the information for managing the medical apparatus 100 on the basis of the transmission information. Data in an arbitrary format, in which various kinds of data related to the medical apparatus 100 is stored, such as a database for maintenance, is exemplified, for example, as the information for managing the medical apparatus 100.

The information processing apparatus 300 determines whether there is data to be updated by comparing various kinds of information included in the transmission information with information that is stored in the information for managing the medical apparatus 100. Data that indicates an apparatus ID, data that indicates a serial number, position information, data that indicates a sales agent, data that indicates an owner who owns the medical apparatus 100, and data that indicates a version of software or the like are exemplified, for example, as the information that is included in the transmission information to be compared.

The information processing apparatus 300 determines that there is data to be updated in a case in which the information that indicates content different from the information that is stored in the information for managing the medical apparatus 100 is included in the transmission information, for example. Note that the method for determining whether there is data to be updated is not limited to the example described above and that the information processing apparatus 300 may determine whether there is data to be updated by an arbitrary algorithm.

Then, the information processing apparatus 300 updates the information for managing the medical apparatus 100 in a case in which it is determined that there is data to be updated. In addition, the information processing apparatus 300 does not update the information for managing the medical apparatus 100 in a case in which it is not determined that there is data to be updated.

Note that the processing in a case in which the type of the content of the notification is determined to be the current state report notification is not limited to the example described above.

For example, the information processing apparatus 300 can also perform processing related to preventive maintenance.

The information processing apparatus 300 determines timing for maintenance of the medical apparatus 100 by processing based on an arbitrary algorithm such as threshold determination processing on the basis of a current state (a use status of parts such as a cumulative operating time and the numbers of times a battery is charged and discharged, and an operation result of a self-diagnosis function, for example) of the medical apparatus 100 that is indicated by the transmission information, for example. Then, the information processing apparatus 300 provides the notification of the determined timing to the contact destination corresponding to the medical apparatus 100 or the supervisor or the operator of the information processing apparatus 300, for example.

Hereinafter, an example of a configuration of the information processing apparatus 300 will be described, and an example of processing related to the processing method in the information processing apparatus 300 will be described.

### [1-3-2] Configuration of Information Processing Apparatus 300

FIG. 6 is a block diagram illustrating an example of a configuration of the information processing apparatus 300 (second information processing apparatus) according to the embodiment. The information processing apparatus 300 includes a communication unit 302 and a control unit 304, for example.

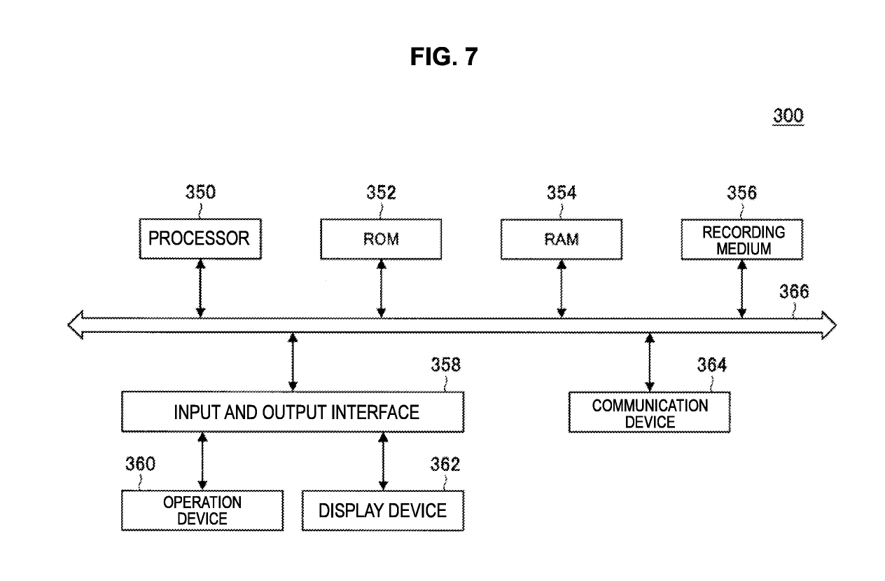

FIG. 7 is an explanatory diagram illustrating an example of a hardware configuration of the information processing apparatus 300 (second information processing apparatus) according to the embodiment. The information processing apparatus 300 includes a processor 350, a ROM 352, a RAM 354, a recording medium 356, an input and output interface 358, an operation device 360, a display device 362, and a communication device 364, for example. In addition, the information processing apparatus 300 connects the respective components by a bus 366 that is a transmission path of data, for example. In addition, the information processing apparatus 300 is driven by electric power supplied from an internal power source such as a battery provided in the information processing apparatus 300, electric power supplied from an external power source connected thereto, or the like, for example.

The processor 350 includes, for example, one or two or more processors that include computation circuits such as an MPU, various processing circuits, and the like and functions as the control unit 304 that controls the entire information processing apparatus 300. In addition, the processor 350 plays roles as a determination unit 310 and the processing unit 312, which will be described later, for example, in the information processing apparatus 300. Note that one of or both the determination unit 310 and the processing unit 312 may include dedicated (or general-purpose) circuits (processors or the like that are separate from the processor 350, for example).

The ROM 352 stores control data such as programs and computation parameters that are used by the processor 350 and the like. The RAM 354 temporarily stores the programs and the like that are executed by the processor 350, for example.

The recording medium 356 functions as a storage unit (not illustrated) and stores various kinds of data such as the data related to the processing method in the information processing apparatus 300 such as “a database (or a table) in which the apparatus ID and the serial number of the medical apparatus 100 are associated with the data that indicates the installation location” and various applications, for example. Here, a magnetic recording medium such as a hard disk and a non-volatile memory such as a flash memory are exemplified, for example, as the recording medium 356. In addition, the recording medium 356 may be able to be attached to and detached from the information processing apparatus 300.

The input and output interface 358 connects the operation device 360 and the display device 362, for example. The operation device 360 functions as an operation unit (not illustrated), and the display device 362 functions as a display unit (not illustrated). Here, a USB terminal, a DVI terminal, an HDMI (registered trademark) terminal, various processing circuits, and the like are exemplified, for example, as the input and output interface 358.

In addition, the operation device 360 is provided on the information processing apparatus 300, for example, and is connected to the input and output interface 358 inside the information processing apparatus 300. A button, a direction key, a rotation-type selector such as a jog dial, a combination thereof, and the like are exemplified, for example, as the operation device 360.

In addition, the display device 362 is provided on the information processing apparatus 300, for example, and is connected to the input and output interface 258 inside the information processing apparatus 300. A liquid crystal display, an organic EL display, and the like are exemplified, for example, as the display device 362.

Note that it is needless to say that the input and output interface 358 can also be connected to an external device such as an external operation device of the information processing apparatus 300 (for example, a keyboard, a mouse, or the like) or an external display device. In addition, the display device 362 may be a device that allows for display and user's operation, such as a touch panel, for example.

The communication device 364 is a communication mechanism that is provided in the information processing apparatus 300 and functions as the communication unit 302 that performs wireless or wired communication with the external apparatus such as the information processing apparatus 200 via a network (or directly). Here, a communication antenna and an RF circuit (wireless communication), an IEEE 802.15.1 port and a transmission and reception circuit (wireless communication), an IEEE 802.11 port and a transmission and reception circuit (wireless communication), a LAN terminal and a transmission and reception circuit (wired communication), and the like are exemplified, for example, as the communication device 364. In addition, the communication device 364 may have an arbitrary configuration that is compatible with the network.

The information processing apparatus 300 performs the processing related to the processing method in the information processing apparatus 300 by the hardware configuration illustrated in FIG. 7, for example. Note that the hardware configuration of the information processing apparatus 300 according to the embodiment is not limited to the configuration illustrated in FIG. 7.

For example, the information processing apparatus 300 may not include the communication device 364 in a case of performing communication with the external apparatus or the like via an external communication device connected thereto. In addition, the communication device 364 may have a configuration capable of performing communication with one or two or more external apparatuses and the like by a plurality of communication schemes.

In addition, the information processing apparatus 300 can employ a configuration in which a part or an entirety of the recording medium 356, the operation device 360, and the display device 362 are not provided, for example.

In addition, the information processing apparatus 300 can employ a configuration in accordance with an application example of the information processing apparatus 300, for example.

In addition, a part or an entirety of the configuration illustrated in FIG. 7 (or configurations related to modification examples), for example, may be realized by one or two or more ICs.

Referring again to FIG. 6, an example of the configuration of the information processing apparatus 300 will be described. The communication unit 302 is a communication mechanism that is provided in the information processing apparatus 300 and performs wireless or wired communication with an external apparatus such as the information processing apparatus 200 via a network (or directly). In addition, the communication of the communication unit 302 is controlled by the control unit 304, for example.

Here, although a communication antenna and an RF circuit, a LAN terminal and a transmission and reception circuit, and the like are exemplified, for example, as the communication unit 302, the configuration of the communication unit 302 is not limited to that described above. For example, the communication unit 302 can employ a configuration that is compatible with an arbitrary standard capable of performing communication with the USB terminal, the transmission and reception circuit, and the like or an arbitrary configuration capable of performing communication with an external apparatus via a network. In addition, the communication unit 302 may have a configuration capable of performing communication with one or two or more external apparatuses and the like by a plurality of communication schemes.

The control unit 304 includes the processor 350, for example, and plays a role in controlling the entire information processing apparatus 300. In addition, the control unit 304 includes the determination unit 310 (second determination unit) and the processing unit 312, for example, and plays a leading role in performing the processing related to the processing method in the information processing apparatus 300.

The determination unit 310 determines the type of the content of the notification in the medical apparatus 100 on the basis of the transmission information in a case in which the transmission information has been acquired. As described above, the determination unit 310 determines that the notification is an error occurrence notification and that the notification is the current state report notification, for example, as the type of the content of the notification. The result of the determination by the determination unit 310 is delivered to the processing unit 312.

The processing unit 312 performs processing corresponding to the type of the notification determined by the determination unit 310. In a case in which it is determined by the determination unit 310 that the type of the content of the notification is the error occurrence notification, for example, the processing unit 312 performs processing of providing a notification that an error has occurred in the medical apparatus 100 to a contact destination corresponding to the specified medical apparatus 100. In addition, in a case in which it is determined by the determination unit 310 that the type of the content of the notification is the current state report notification, for example, the processing unit 312 performs one of or both processing of updating the information for managing the medical apparatus 100 and processing related to preventive maintenance on the basis of the transmission information.

In a case in which the processing corresponding to the determined type of the notification has been performed, the processing unit 312 may cause the communication unit 302 to transmit response information corresponding to the performed processing to the information processing apparatus 200 that has transmitted the transmission information. The processing unit 312 causes the response information to be transmitted by the same method as that of the transmission method by which the information processing apparatus 200 transmits the transmission information or a method that is different from the transmission method by which the information processing apparatus 200 transmits the transmission information, for example.

The information processing apparatus 300 determines the type of the content of the notification in the medical apparatus 100 on the basis of the acquired transmission information and performs processing corresponding to the determined type of the notification with the configuration illustrated in FIG. 6, for example.

Note that the configuration of the information processing apparatus 300 is not limited to the example illustrated in FIG. 6.

For example, the information processing apparatus 300 can include (realize by other processing circuits, for example) one of or both the determination unit 310 and the processing unit 312 illustrated in FIG. 6 separately from the control unit 304.

In addition, the information processing apparatus 300 may not include the communication unit 302 in a case in which the information processing apparatus 300 performs communication with an external apparatus via an external communication device that has functions and a configuration similar to those of the communication unit 302, for example.

### [2] Use Case Using Medical Information Processing System

Next, a use case that is realized by using the aforementioned medical information processing system 1000 will be described. Note that it is needless to say that the use case that is realized by using the medical information processing system 1000 is not limited to the examples described below.

### [2-1] First Example of Use Case: Use Case when Error has Occurred in Medical Apparatus 100

If an error has occurred in the medical apparatus 100, a two-dimensional code (the reference numeral N illustrated in FIG. 1, for example) is displayed on the display screen of the medical apparatus 100. The user of the information processing apparatus 200 captures an image of the two-dimensional code displayed on the display screen by the imaging device 266 provided in the information processing apparatus 200.

The following (A) to (C) are realized by the image of the two-dimensional code being captured by the information processing apparatus 200.

(A) Content of the error and a strategy for handling the error are displayed on the display screen of the display device 262 that is provided in the information processing apparatus 200.

(B) The transmission information is transmitted from the information processing apparatus 200 to the information processing apparatus 300 that is provided in a maintenance center of a manufacturer of the medical apparatus 100, and the information processing apparatus 300 automatically provides a request for repairing the medical apparatus 100. The manufacture specifies a factor of the error and specifies parts that request replacing or repairing on the basis of the content indicated by the transmission information that is transmitted from the information processing apparatus 200 on the basis of the notification of the medical apparatus 100, that is, on the basis of the accurate content of the notification obtained from the medical apparatus 100. Therefore, the manufacturer can quickly and appropriately handle the error.

(C) The processing performed by the information processing apparatus 200 is performed by the processor 250 that is provided in the information processing apparatus 200, for example, executing application software (program) for performing the processing related to the processing method in the information processing apparatus 200. In a case in which the processing performed by the information processing apparatus 200 is performed by executing the application software described above, the user of the information processing apparatus 200 can perform communication necessary to handle the error, such as a reply and additional inquiry from the manufacturer, via the application software described above. In addition, in a case in which the processing performed by the information processing apparatus 200 is performed by executing the application software described above, the user of the information processing apparatus 200 can refer to a history of handling the error via the application software.

### [2-2] Second Example of Use Case: Use Case when Maintenance of Medical Apparatus 100 is Performed

If the user of the information processing apparatus 200 performs an operation for the maintenance by using the operation device that is provided in the medical apparatus 100 at an arbitrary timing such as at the time of checking before start of work, the two-dimensional code (the reference numeral N illustrated in FIG. 1, for example) is displayed on the display screen of the medical apparatus 100. The user of the information processing apparatus 200 captures an image of the two-dimensional code displayed on the display screen by the imaging device 266 that is provided in the information processing apparatus 200.

The following (a) to (c) are realized by the image of the two-dimensional code being captured by the information processing apparatus 200.

(a) Since the information processing apparatus 200 can obtain precise content of the notification from the medical apparatus 100, the user of the information processing apparatus 200 can precisely and easily check the medical apparatus 100. In addition, the result of the checking is automatically recorded in a recording medium by the information processing apparatus 200 recording the information corresponding to the result of the determination acquired from the medical apparatus 100 in a recording medium such as a storage unit (which will be described later).

(b) The transmission information is transmitted from the information processing apparatus 200 to the information processing apparatus 300 that is provided in the maintenance center of the manufacturer of the medical apparatus 100, and the information processing apparatus 300 performs the processing related to preventive maintenance on the basis of the transmission information. Therefore, the manufacturer can recognize presage of potential malfunction of the medical apparatus 100 and take a preventive countermeasure. In addition, the manufacture can recognize a timing at which a consumable part is to be replaced in the medical apparatus 100 in advance and quickly and appropriately take a measure such as order or replacement.

(c) The transmission information is transmitted from the information processing apparatus 200 to the information processing apparatus 300 that is provided in the maintenance center of the manufacturer of the medical apparatus 100, and processing of updating the information for managing the medical apparatus 100 on the basis of the transmission information is performed in the information processing apparatus 300. Therefore, the manufacturer can collect and manage a variety of kinds of information that can be used for managing the medical apparatus 100, such as an apparatus ID, a serial number, an installation location, and a version of software. In addition, a variety of information that can be used for managing the medical apparatus 100 is updated to the latest information by the information processing apparatus 300 executing the processing of updating the information for managing the medical apparatus 100.

### [3] Example of Effects that are Achieved by Using Medical Information Processing System According to Embodiment

The following effects, for example, are achieved by the medical information processing system according to the embodiment being used. Note that it is needless to say that the effects achieved by the medical information processing system according to the embodiment being used are not limited to the examples described below.
A person who is not familiar with the medical apparatus 100 can also easily take a countermeasure against an error in the medical apparatus 100 and perform checking for maintenance. In addition, it is not necessary to take efforts for reporting the situation in detail when the error has occurred in the medical apparatus 100. Therefore, it is possible to improve convenience for the person who uses the medical apparatus 100.
The transmission information automatically includes information that is useful for the strategy for handling the error by the facility, the sales agent, or the like by the information processing apparatus 200 transmitting the transmission information that includes the additional information.
Persons who are in the installation location of the medical apparatus 100 can share a situation of malfunction in the medical apparatus 100 by sharing a history of the countermeasure against the error in the information processing apparatus 200.
Since the medical apparatus 100 performs the processing related to the processing method in the medical apparatus 100 by the processor (not illustrated), for example, it is not necessary to provide, in the medical apparatus 100, special hardware for executing the processing related to the processing method in the medical apparatus 100.
In a case in which the medical apparatus 100 provides the notification of the information corresponding to the result of the determination by causing the information to be displayed on the display screen, or in a case in which the medical apparatus 100 provides the notification of the information corresponding to the result of the determination by the one-way communication, there is no risk that the medical apparatus 100 is improperly accessed by the outside due to the method of providing the notification.
The notifications of detailed content of the error that has occurred in the medical apparatus 100 and how to handle the error can be provided to the user of the information processing apparatus 200 by the processing of analyzing the medical apparatus notification information acquired from the medical apparatus 100 being performed by the information processing apparatus 200.
If the medical apparatus 100 has a display device capable of displaying characters, such as one mounted on a calculator, it is possible to provide a notification of the information corresponding to the result of the determination to be displayed on the display screen. Therefore, it is possible to use a variety of medical apparatuses as the medical apparatus 100.
The information processing apparatus 200 specifies a primary strategy for handling the error with reference to information that indicates the primary strategy for handling the error, which has been stored in an external apparatus, such as a server, for example. Therefore, the information processing apparatus 200 can specify the primary strategy for handling the error with reference to the information that is appropriately added and updated to the latest content.
The user of the information processing apparatus 200 can perform communication necessary to handle the error, such as a reply and additional inquiry from the manufacturer, via application software for performing the processing related to the processing method in the information processing apparatus 200. Therefore, the user of the information processing apparatus 200 can reliably reply to a result of inspection and the like from the manufacturer through the application software described above.
In a case in which the medical apparatus 100 provides the notification of the information corresponding to the result of the determination by acoustic waves, the information processing apparatus 200 can deliver the content of the notification provided by the medical apparatus 100 to the manufacturer of the medical apparatus 100 even in a state in which the information processing apparatus 200 is in a pocket or the like of the user of the information processing apparatus 200.
It is possible to precisely and easily perform the checking for maintenance of the medical apparatus 100 at an arbitrary timing such as such as at the time of checking before start of work while omitting efforts of the user of the information processing apparatus 200. In addition, the result of the checking can be automatically recorded in a recording medium by the information processing apparatus 200 recording the information corresponding to the result of the determination acquired from the medical apparatus 100 in the recording medium such as a storage unit (which will be described later).

### Program According to Embodiment

### [I] Program for Causing Functions of Medical Apparatus 100 According to Embodiment to be Realized

It is possible to improve convenience of the person who uses the medical apparatus 100 by a program for causing a computer system to function as the medical apparatus 100 (a program that causes the computer system to function as the determination unit 110 and the notification processing unit 112 illustrated in FIG. 2, for example) being executed by a processor or the like in the computer system. Here, a single computer or a plurality of computers are exemplified as the computer system according to the embodiment. A series of processing related to the processing method in the medical apparatus 100 according to the embodiment is performed by the computer system according to the embodiment.

In addition, the aforementioned effects that are achieved by the processing related to the processing method in the medical observation apparatus according to the embodiment can be achieved by the program that causes the computer system to function as the medical apparatus 100 being executed by the processor or the like in the computer system.

### [II] Program for Causing Functions of Information Processing Apparatus 200 (First Information Processing Apparatus) According to Embodiment to be Realized

It is possible to improve convenience for the person who uses the medical apparatus 100 by a program that causes a computer system to function as the information processing apparatus 200 (a program that causes the computer system to function as “the acquisition processing unit 210 and the transmission processing unit 212”, “the acquisition processing unit 210, the transmission processing unit 212, and the analysis unit 214”, “the acquisition processing unit 210, the transmission processing unit 212, and the notification processing unit 216”, or “the acquisition processing unit 210, the transmission processing unit 212, the analysis unit 214, and the notification processing unit 216” illustrated in FIG. 3, for example) being executed by a processor or the like in the computer system. A series of processing related to the processing method in the information processing apparatus 200 is performed by the computer system.

In addition, the aforementioned effects achieved by the processing related to the processing method in the information processing apparatus 200 can be achieved by the program that causes the computer system to function as the information processing apparatus 200 being executed by a processor or the like in the computer system.

### [III] Program for Causing Functions of Information Processing Apparatus 300 (Second Information Processing Apparatus) According to Embodiment to be Realized

It is possible to improve the convenience for the person who uses the medical apparatus 100 by a program that causes a computer system to function as the information processing apparatus 300 (a program that causes the computer system to function as the determination unit 310 and the processing unit 312 illustrated in FIG. 6, for example) being executed by a processor or the like in the computer system. A series of processing related to the processing method in the information processing apparatus 300 is performed by the computer system.

In addition, the aforementioned effects achieved by the processing related to the processing method in the information processing apparatus 300 can be achieved by the program for causing the computer system to function as the information processing apparatus 300 being executed by the processor or the like in the computer system.

It should be understood by those skilled in the art that various modifications, combinations, sub-combinations and alterations may occur depending on design requirements and other factors insofar as they are within the scope of the appended claims or the equivalents thereof.

For example, although the above description has illustrated that the programs (computer programs) for causing computer systems to function as the medical apparatus 100 according to the embodiment, the information processing apparatus 200 (first information processing apparatus) according to the embodiment, and the information processing apparatus 300 (second information processing apparatus) according to the embodiment, respectively, are provided, the embodiment can further provide recording mediums that store the programs described above, respectively, or recording mediums that store two or more of the programs described above together.

The aforementioned configuration illustrates an example of the embodiment, and it is a matter of course that the configuration also belongs to the technical scope of the present disclosure.

Further, the effects described in this specification are merely illustrative or exemplified effects, and are not limitative. That is, with or in the place of the above effects, the technology according to an embodiment of the present disclosure may achieve other effects that are clear to those skilled in the art from the description of this specification.

Additionally, the present technology may also be configured as below.

(1) A medical apparatus including:

a determination unit that determines a state that is a target of a notification; and

a notification processing unit that causes, on a basis of a result of the determination, notifications of apparatus management information and medical apparatus notification information to be provided in a format in which an external apparatus is able to read the information, the apparatus management information being used for managing the medical apparatus, the medical apparatus notification information indicating content of a notification in the medical apparatus.

(2) The medical apparatus according to (1),

in which the determination unit determines an error notification state in which a notification that an error has occurred is to be provided, as the state that is a target of a notification, and

the notification processing unit causes the notification of the medical apparatus notification information to be provided, the medical apparatus notification information indicating content of the error that has occurred.

(3) The medical apparatus according to (1) or (2),

in which the determination unit determines a current notification state in which a notification of a current state of the medical apparatus is to be provided as the state that is a target of notification, and

the notification processing unit causes the notification of the medical apparatus notification information to be provided, the medical apparatus notification information indicating the current state of the medical apparatus.

(4) The medical apparatus according to any one of (1) to (3), in which the notification processing unit causes the apparatus management information and the medical apparatus notification information to be displayed on a display screen, thereby causing the notifications of the apparatus management information and the medical apparatus notification information to be provided.
(5) The medical apparatus according to any one of (1) to (4), in which the notification processing unit causes the notifications of the apparatus management information and the medical apparatus notification information to be provided by one-way communication.
(6) The medical apparatus according to (5), in which the notification processing unit causes a sound output device to output a signal obtained by modulating the apparatus management information and the medical apparatus notification information, thereby causing the notifications of the apparatus management information and the medical apparatus notification information to be provided.
(7) The medical apparatus according to any one of (1) to (6), in which the notification processing unit encrypts one of or both the apparatus management information and the medical apparatus notification information and causes the notifications of the apparatus management information and the medical apparatus notification information to be provided.
(8) An information processing apparatus including:

an acquisition processing unit that performs processing of acquiring apparatus management information that is used for managing a medical apparatus and medical apparatus notification information that indicates content of a notification in the medical apparatus from the medical apparatus; and

a transmission control unit that causes transmission information to be transmitted to a set predetermined transmission destination, the transmission information including the acquired apparatus management information and information based on the acquired medical apparatus notification information.

(9) The information processing apparatus according to (8), in which the transmission control unit causes the transmission information to be transmitted, the transmission information further including additional information to be added by the information processing apparatus.
(10) The information processing apparatus according to (8) or (9), further including:

an analysis unit that analyzes the acquired medical apparatus notification information,

in which information based on the medical apparatus notification information included in the transmission information is information that indicates a result of the analysis of the acquired medical apparatus notification information.

(11) The information processing apparatus according to (10), further including:

a notification processing unit that causes a notification of information to be provided, the information being based on the result of the analysis of the acquired medical apparatus notification information.

(12) The information processing apparatus according to any one of (8) to (11), further including:

a notification processing unit that causes a notification of response information to be provided, the response information indicating a response to the transmission information from the predetermined transmission destination.

(13) The information processing apparatus according to any one of (8) to (12), in which information based on the medical apparatus notification information included in the transmission information is the acquired medical apparatus notification information.
(14) An information processing apparatus including:

a determination unit that determines a type of content of a notification in a medical apparatus on a basis of transmission information that includes apparatus management information that is used for managing the medical apparatus and information based on medical apparatus notification information that indicates content of the notification in the medical apparatus in a case in which the transmission information is acquired; and

a processing unit that performs processing corresponding to the determined type of the notification.

(15) The information processing apparatus according to (14), in which the determination unit determines whether the type of the content of the notification is an error occurrence notification in which a notification that an error has occurred in the medical apparatus is to be provided or a current state report notification in which a notification about a current state of the medical apparatus is to be provided.
(16) A processing method that is executed by a medical apparatus, the processing method including:

determining a state that is a target of a notification; and

causing, on a basis of a result of the determination, notifications of apparatus management information and medical apparatus notification information to be provided in a format in which an external apparatus is able to read the information, the apparatus management information being used for managing the medical apparatus, the medical apparatus notification information indicating content of a notification in the medical apparatus.

(17) A processing method that is executed by an information processing apparatus, the processing method including:

acquiring apparatus management information that is used for managing a medical apparatus and medical apparatus notification information that indicates content of a notification in the medical apparatus from the medical apparatus; and

causing transmission information to be transmitted to a set predetermined transmission destination, the transmission information including the acquired apparatus management information and information based on the acquired medical apparatus notification information.

(18) A processing method that is executed by an information processing apparatus, the processing method including:

determining a type of content of a notification in a medical apparatus on a basis of transmission information that includes apparatus management information that is used for managing the medical apparatus and information based on medical apparatus notification information that indicates the content of the notification in the medical apparatus in a case in which the transmission information is acquired; and

performing processing corresponding to the determined type of the notification.

(19) A program for causing a computer to realize:

a function of acquiring apparatus management information that is used for managing a medical apparatus and medical apparatus notification information that indicates content of a notification in the medical apparatus form the medical apparatus; and

a function of causing transmission information to be transmitted to a set predetermined transmission destination, the transmission information including the acquired apparatus management information and information based on the acquired medical apparatus notification information.

(20) A medical information processing system including:

a medical apparatus;

a first information processing apparatus; and

a second information processing apparatus,

in which the medical apparatus includes
a first determination unit that determines a state that is a target of a notification, and
a notification processing unit that causes, on a basis of a result of the determination, notifications of apparatus management information and medical apparatus notification information to be provided in a format in which an external apparatus is able to read the information, the apparatus management information being used for managing the medical apparatus, the medical apparatus notification information indicating content of a notification in the medical apparatus,

the first information processing apparatus includes
an acquisition processing unit that performs processing of acquiring the apparatus management information and the medical apparatus notification information from the medical apparatus, and
a transmission control unit that causes transmission information to be transmitted to a set predetermined transmission destination, the transmission information including the acquired apparatus management information and information based on the acquired medical apparatus notification information, and

the second information processing apparatus includes
a second determination unit that determines a type of the content of the notification in the medical apparatus on a basis of the transmission information in a case in which the transmission information is acquired, and
a processing unit that performs processing corresponding to the determined type of the notification.

What is claimed is:

1. A medical apparatus comprising:
a determination unit that determines a state that is a target of a notification; and
a notification processing unit that causes, on a basis of a result of the determination, notifications of apparatus management information and medical apparatus notification information to be provided in a format in which an external apparatus is able to read the information, the apparatus management information being used for managing the medical apparatus, the medical apparatus notification information indicating content of a notification in the medical apparatus.

2. The medical apparatus according to claim 1,
wherein the determination unit determines an error notification state in which a notification that an error has occurred is to be provided, as the state that is a target of a notification, and
the notification processing unit causes the notification of the medical apparatus notification information to be provided, the medical apparatus notification information indicating content of the error that has occurred.

3. The medical apparatus according to claim 1,
wherein the determination unit determines a current notification state in which a notification of a current state of the medical apparatus is to be provided as the state that is a target of notification, and
the notification processing unit causes the notification of the medical apparatus notification information to be provided, the medical apparatus notification information indicating the current state of the medical apparatus.

4. The medical apparatus according to claim 1, wherein the notification processing unit causes the apparatus management information and the medical apparatus notification information to be displayed on a display screen, thereby causing the notifications of the apparatus management information and the medical apparatus notification information to be provided.

5. The medical apparatus according to claim 1, wherein the notification processing unit causes the notifications of the apparatus management information and the medical apparatus notification information to be provided by one-way communication.

6. The medical apparatus according to claim 5, wherein the notification processing unit causes a sound output device to output a signal obtained by modulating the apparatus management information and the medical apparatus notification information, thereby causing the notifications of the apparatus management information and the medical apparatus notification information to be provided.

7. The medical apparatus according to claim 1, wherein the notification processing unit encrypts one of or both the apparatus management information and the medical apparatus notification information and causes the notifications of the apparatus management information and the medical apparatus notification information to be provided.

8. An information processing apparatus comprising:
an acquisition processing unit that performs processing of acquiring apparatus management information that is used for managing a medical apparatus and medical apparatus notification information that indicates content of a notification in the medical apparatus from the medical apparatus; and
a transmission processing unit that causes transmission information to be transmitted to a set predetermined transmission destination, the transmission information including the acquired apparatus management information and information based on the acquired medical apparatus notification information.

9. The information processing apparatus according to claim 8, wherein the transmission processing unit causes the transmission information to be transmitted, the transmission information further including additional information to be added by the information processing apparatus.

10. The information processing apparatus according to claim 8, further comprising:
an analysis unit that analyzes the acquired medical apparatus notification information,
wherein information based on the medical apparatus notification information included in the transmission information is information that indicates a result of the analysis of the acquired medical apparatus notification information.

11. The information processing apparatus according to claim 10, further comprising:
a notification processing unit that causes a notification of information to be provided, the information being based on the result of the analysis of the acquired medical apparatus notification information.

12. The information processing apparatus according to claim 8, further comprising:
a notification processing unit that causes a notification of response information to be provided, the response information indicating a response to the transmission information from the predetermined transmission destination.

13. The information processing apparatus according to claim 8, wherein information based on the medical apparatus notification information included in the transmission information is the acquired medical apparatus notification information.

14. A medical information processing system comprising:
a medical apparatus;
a first information processing apparatus; and
a second information processing apparatus,
wherein the medical apparatus includes
a first determination unit that determines a state that is a target of a notification, and
a notification processing unit that causes, on a basis of a result of the determination, notifications of apparatus management information and medical apparatus notification information to be provided in a format in which an external apparatus is able to read the information, the apparatus management information being used for managing the medical apparatus, the medical apparatus notification information indicating content of a notification in the medical apparatus,
the first information processing apparatus includes
an acquisition processing unit that performs processing of acquiring the apparatus management information and the medical apparatus notification information from the medical apparatus, and
a transmission processing unit that causes transmission information to be transmitted to a set predetermined transmission destination, the transmission information including the acquired apparatus management information and information based on the acquired medical apparatus notification information, and
the second information processing apparatus includes
a second determination unit that determines a type of the content of the notification in the medical apparatus on a basis of the transmission information in a case in which the transmission information is acquired, and
a processing unit that performs processing corresponding to the determined type of the notification.
